(19) United States
(12) Patent Application Publication
(10) Pub. No.: US 2022/0184354 A1
(43) Pub. Date: Jun. 16, 2022

(54) TREATMENT DEVICE AND TREATMENT METHOD

(71) Applicant: TERUMO KABUSHIKI KAISHA, Tokyo (JP)

(72) Inventor: NAOKI ISHII, Kanagawa (JP)

(21) Appl. No.: 17/685,591

(22) Filed: Mar. 3, 2022

Related U.S. Application Data
(63) Continuation of application No. PCT/JP2020/036356, filed on Sep. 25, 2020.

(30) Foreign Application Priority Data
Sep. 27, 2019 (JP) 2019-176920

(51) Int. Cl. A61M 25/10; A61B 17/22

(52) U.S. Cl. CPC A61M 25/104; A61B 17/22; A61B 2017/22062; A61M 2025/105; A61M 2025/1052; A61M 2205/0266; A61B 2017/22069

(57) ABSTRACT

A treatment device and a treatment method are capable of improving permeability of a physiologically active substance into a blood vessel wall while maintaining a blood flow. A treatment device for treating a lesion in a blood vessel includes: a shaft portion including at least one lumen; a balloon disposed at a distal side of the shaft portion and configured to inflate; and at least one anchor member that can be inserted into the lumen and to radially expand at a state in which a distal portion protrudes from the lumen. The balloon has an outer diameter when inflated that is smaller than an outer diameter of the anchor member when radially expanded. When the anchor member radially expands in a blood vessel to come into contact with a blood vessel wall, the balloon is held away from the blood vessel wall.

### CROSS REFERENCE TO RELATED APPLICATIONS

The present application is a continuation of and claims benefit to PCT Application No. PCT/JP2020/036356 filed on Sep. 25, 2020, entitled “TREATMENT DEVICE AND TREATMENT METHOD” which claims priority to Japanese Patent Application No. 2019-176920 filed on Sep. 27, 2019. The entire disclosure of the applications listed above are hereby incorporated herein by reference, in their entirety, for all that they teach and for all purposes.

### FIELD

The present disclosure relates to a treatment device and a treatment method used in a transvascular manner.

### BACKGROUND

Coronary artery bypass graft (CABG) was developed by Bailey-Hirose, Garrett, Favaloro et al. from 1966 to 1968 as a treatment method for coronary artery diseases such as angina pectoris, acute myocardial infarction (AMI), and the like. However, CABG, which is a thoracotomy, is highly invasive to patients, requires a long time from a hospitalization period to society recovery, and incurs a high medical expense.

Percutaneous old balloon angioplasty (POBA) was developed by Gruenzig et al. in 1977. POBA has been proven effective and safe by a large number of clinical trials, and has been widely applied together with CABG as a treatment method for ischemic heart diseases. POBA is less invasive to patients and is an economical treatment method, but causes a new problem that acute coronary obstruction occurs at a frequency of 3% to 5%, and remote restenosis occurs at a frequency of 30% to 50%. In order to overcome such problem of POBA, bare metal stent (BMS) was developed, and was clinically used for humans for the first time by Sigwart et al. in 1986. After that, the stent became commercially available as a Palmaz-Shatz® (registered trademark) stent through improvement in shape, improvement in delivery catheter, and the like. Regarding a preventive effect on restenosis of BMS, a large-scale multicenter randomized trial (BENESTENT-1) was performed in 1994. However, with regard to remote restenosis, an occurrence of about 20% was still observed, which has not been solved even by BMS.

A drug eluting stent (DES) was developed with an aim for solving this remaining remote restenosis. In the latter half of the 1990s, the Cypher® (registered trademark) stent using sirolimus (a macrolide immunosuppressant) was developed by Johnson & Johnson (Cordis Corporation); and the TAXUS® (registered trademark) stent using paclitaxel (an anticancer agent) was developed by Boston Scientific Corporation. These stents generally have a surface coated with a non-absorbable polymer impregnated with a drug. The impregnated drug is slowly released from a polymer layer and diffuses into an indwelling blood vessel to prevent smooth muscle proliferation, which is a cause of the restenosis disease state.

As a result of the development of the drug elution stent, a restenosis rate of coronary artery has been dramatically controlled. However, a site of a myocardium exposed to an ischemic state, particularly upon onset of acute myocardial infarction (AMI), has been damaged, albeit temporarily. As a result, the damaged site may cause a decrease in expansion/contraction function over time. This leads to a new problem of gradual progressing of a decrease in cardiac function and onset of heart failure.

Initially, in a procedure of percutaneous coronary intervention (PCI) for AMI, it is of the highest priority to expand and open an infarction site as soon as possible, and reopen blood flow to ensure the blood flow to the entire body, thereby saving the life of the patient. For this reason, it is a main object to perform treatment on the infarction site (reopening of blood flow), while damage to a downstream myocardium of the infarction site has hardly been considered.

### SUMMARY

The present disclosure is made to solve the above problems, and an object of the present disclosure is to provide a treatment device and a treatment method capable of improving permeability of a physiologically active substance into a blood vessel wall while maintaining a blood flow.

The treatment device according to the present disclosure for achieving the above object is a treatment device for treating a lesion in a blood vessel. The treatment device includes: a shaft portion including at least one lumen; a balloon disposed at a distal side of the shaft portion and configured to inflate; and at least one anchor member configured to be inserted into the lumen and to radially expand at a state in which a distal portion protrudes from the lumen. The balloon has an outer diameter when inflated that is smaller than an outer diameter of the anchor member when radially expanded. When the anchor member radially expands in a blood vessel to come into contact with a blood vessel wall, the balloon is held away from the blood vessel wall.

A treatment method according to the disclosure for achieving the above object is a treatment method for causing a physiologically active substance to act on a blood vessel wall in a blood vessel. The treatment method includes: an inflation step of inflating an inflatable inflation body in the blood vessel to dispose the inflation body at a position away from the blood vessel wall; and a guiding step of releasing the physiologically active substance from an upstream side of the blood vessel relative to the inflation body, thereby guiding the physiologically active substance toward the blood vessel wall by the inflation body.

In the treatment device configured as described above, when inflated, the balloon can be maintained at a substantially central portion in the blood vessel without coming into contact with the blood vessel wall by causing the anchor member to come into contact with the blood vessel wall when radially expanded. As a result, the treatment device can cause the balloon to guide the physiologically active substance released from upstream of the balloon to the vicinity of the blood vessel wall while maintaining the blood flow. Accordingly, the physiologically active substance released into the blood vessel can be carried by the blood flow to flow to the vicinity of the blood vessel wall, which has a high shearing stress and is advantageous for taking in the substance. Therefore, the treatment device can effectively guide the physiologically active substance to the vicinity of the blood vessel wall, thereby improving permeability of the physiologically active substance into the blood vessel wall.

The anchor member may include at least one anchor member formed of a shape memory alloy. As a result, the anchor member can be radially expanded by being restored to a memorized shape.

The anchor member may include at least one anchor member that is an anchor balloon configured to inflate upon inflow of a fluid. As a result, the anchor member can be radially expanded by the inflow of fluid.

The anchor member may be disposed at a distal side and/or a proximal side of the balloon. As a result, the anchor member can maintain the balloon at the substantially central portion in the blood vessel when inflated, without causing the balloon to come into contact with the blood vessel wall or inhibiting the inflation of the balloon.

The anchor member may include at least one anchor member disposed radially outward of the balloon. The anchor member disposed radially outward of the balloon can effectively prevent the balloon from coming into contact with the blood vessel wall when inflated and maintain the balloon at the substantially central portion in the blood vessel with high accuracy.

The lumen may include at least one lumen passing through a center portion of the balloon and opened in the distal side of the balloon. As a result, the anchor member passing through the lumen opening in the distal side of the balloon can be radially expanded at the distal side of the balloon.

The lumen may include at least one lumen extending along an axis of the balloon and opened in a proximal side of the balloon. As a result, the anchor member passing through the lumen opening in the proximal side of the balloon can be radially expanded at the proximal side of the balloon.

The outer diameter of the balloon may be less than 4 millimeters (mm) when inflated. As a result, even if the balloon is inflated inside a coronary artery, which has an inner diameter of about 4 mm, the balloon is unlikely to come into contact with a blood vessel wall of the coronary artery, and thus is suitable for treatment of coronary artery.

The treatment method configured as described above can maintain the blood flow because the inflation body does not block the blood vessel. The physiologically active substance released from upstream of the inflation body can be guided to the vicinity of the blood vessel wall by the inflation body, and thus the physiologically active substance can be carried by the blood flow to flow to the vicinity of the blood vessel wall, which has a high shearing stress and is advantageous for taking in the substance. Therefore, the treatment method can effectively guide the physiologically active substance to the vicinity of the blood vessel wall while maintaining the blood flow, thereby improving the permeability of the physiologically active substance to the blood vessel wall.

In the treatment method, the blood vessel may be subjected to a treatment for expanding a lesion area in which stenosis or occlusion has occurred. The treatment method may further include a disposition step of radially expanding at least one anchor member capable of radially expanding to an outer diameter larger than that of the inflated inflation body when inflated, and disposing the at least one anchor member on a blood vessel wall upstream of and/or downstream of the lesion area in the blood vessel. As a result, in the treatment method, the anchor member can be disposed on the blood vessel wall at a position without inhibiting the inflation of the inflation body. Further, since the used anchor member has an outer diameter larger than that of the outer diameter of the inflation body, the inflated inflation body can be maintained at the substantially central portion of the blood vessel without coming into contact with the blood vessel wall.

The inflation step may be performed after the disposition step. As a result, the inflation body can be inflated in a state in which the inflation body is held at an appropriate position by the disposition step. Accordingly, when the inflation body is inflated, the inflation body can be maintained at the substantially central portion of the blood vessel with high accuracy without coming into contact with the blood vessel wall.

In the disposition step of the treatment method, an axis of the inflation body may be aligned with a central axis of the blood vessel by expanding the anchor member. As a result, when inflated, the balloon can be maintained at a substantially central portion in the blood vessel with high accuracy without coming into contact with the blood vessel wall.

In the guiding step of the treatment method, the inflation body may be held, or positioned, so as to not come into contact with the blood vessel wall. If the inflation body comes into contact with the blood vessel wall, the physiologically active substance cannot be guided to a part of the blood vessel wall, but by holding the inflation body so as to not come into contact with the blood vessel wall, the physiologically active substance can be effectively guided toward the blood vessel wall.

In the guiding step of the treatment method, the physiologically active substance may be guided toward the blood vessel wall without blocking the blood flow by the inflation body. As a result, since the blood vessel is not blocked by the inflation body, the treatment method can prevent the downstream side from being in ischemic state, thereby improving safety.

### BRIEF DESCRIPTION OF THE DRAWINGS

FIG. 1 is a plan view illustrating a treatment device in accordance with embodiments of the present disclosure;

FIG. 2 is a cross-sectional view illustrating an expansion catheter in accordance with embodiments of the present disclosure;

FIG. 3A is a diagram illustrating a plan view of a distal portion of a first anchor member in accordance with embodiments of the present disclosure;

FIG. 3B is a cross-sectional view along a line A-A of the first anchor member of FIG. 3A in accordance with embodiments of the present disclosure;

FIG. 4A is a diagram illustrating a plan view of a distal portion of a second anchor member in accordance with embodiments of the present disclosure;

FIG. 4B is a cross-sectional view along a line B-B of the second anchor member of FIG. 4A in accordance with embodiments of the present disclosure;

FIG. 5 is a diagram illustrating an area of a balloon and an area surrounded by an anchor member in a cross section orthogonal to an axis of the balloon in accordance with embodiments of the present disclosure;

FIG. 6A illustrates a state of a treatment method in which a catheter is inserted into a blood vessel in accordance with embodiments of the present disclosure;

FIG. 6B illustrates a state of the treatment method in which the treatment device protrudes from the catheter in accordance with embodiments of the present disclosure;

FIG. 6C illustrates a state of the treatment method in which the treatment device is held by the second anchor member in accordance with embodiments of the present disclosure;

FIG. 6D illustrates a state of the treatment method in which the treatment device is held by the first anchor member and the second anchor member in accordance with embodiments of the present disclosure;

FIG. 6E illustrates a state of the treatment method in which a physiologically active substance is guided toward the blood vessel by the treatment device in accordance with embodiments of the present disclosure;

FIG. 7 is a plan view illustrating a first modification of the treatment device in accordance with embodiments of the present disclosure;

FIG. 8 is a plan view illustrating a second modification of the treatment device in accordance with embodiments of the present disclosure;

FIG. 9A is a plan view illustrating a third modification of the treatment device in accordance with embodiments of the present disclosure;

FIG. 9B is a front view seen from a distal side of the third modification of the treatment tool of FIG. 9A in accordance with embodiments of the present disclosure;

FIG. 10A is a plan view of a fourth modification of the treatment device in accordance with embodiments of the present disclosure;

FIG. 10B is a front view seen from the distal side of the fourth modification of the treatment tool of FIG. 10A in accordance with embodiments of the present disclosure;

FIG. 11A illustrates a fifth modification of the treatment device in accordance with embodiments of the present disclosure;

FIG. 11B illustrates a sixth modification of the treatment device in accordance with embodiments of the present disclosure;

FIG. 11C illustrates a seventh modification of the treatment device in accordance with embodiments of the present disclosure;

FIG. 12A illustrates an eighth modification of the treatment device in accordance with embodiments of the present disclosure;

FIG. 12B illustrates a ninth modification of the treatment device in accordance with embodiments of the present disclosure; and

FIG. 13 is a plan view illustrating a tenth modification of the treatment device in accordance with embodiments of the present disclosure.

### DETAILED DESCRIPTION

Hereinafter, embodiments of the present disclosure will be described with reference to drawings. Note that dimensional ratios in the drawings are exaggerated for convenience of description and may differ from actual ratios. Further, in the present description and the drawings, structural elements that have substantially the same function are denoted with the same reference numerals, and repeated explanation of these structural elements is omitted. In the present description, a side to be inserted into a blood vessel of a device is referred to as a “distal side”, and a hand-side for operation is referred to as a “proximal side”.

A treatment device 10 according to at least one embodiment of the present disclosure is a device for treatment to be performed subsequently to percutaneous coronary intervention (PCI) performed for treatment of acute myocardial infarction (AMI). After PCI is performed to expand and open a lesion area where stenosis or occlusion has occurred, the treatment device 10 can cause a physiologically active substance to act on a damaged blood vessel, myocardium, and the like via an inner surface of a peripheral blood vessel including the lesion area, which is damaged by PCI and thus has an improved substance permeability, and gaps generated between vascular endothelial cells.

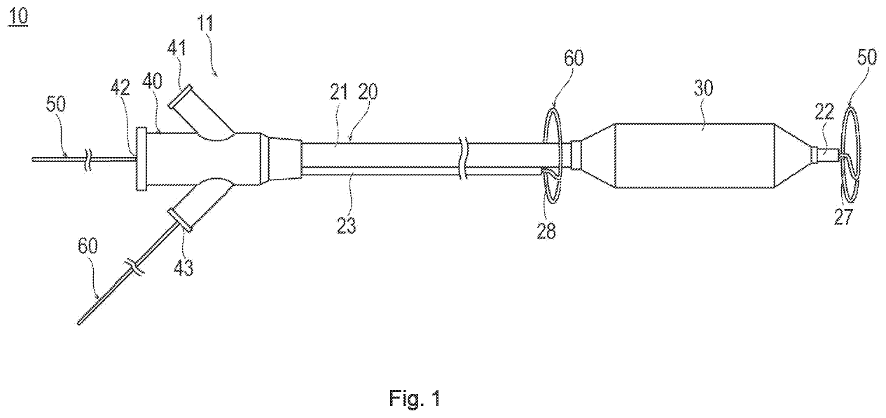

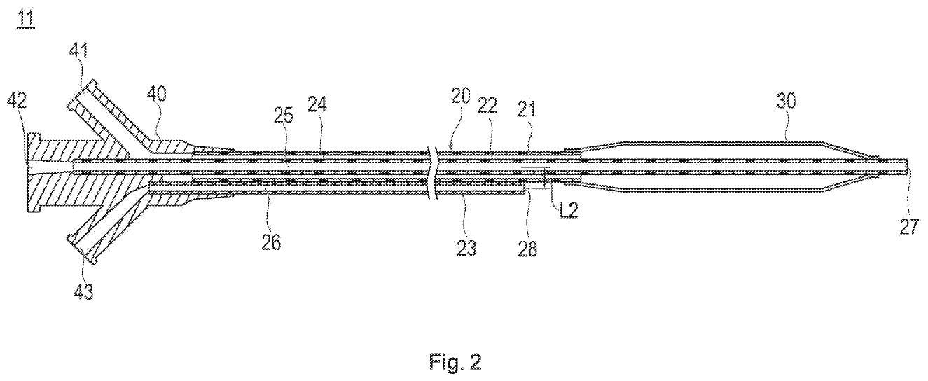

First, a configuration of the treatment device 10 will be described. As illustrated in FIGS. 1 and 2, the treatment device 10 includes an expandable catheter 11 including an inflatable balloon 30, and a first anchor member 50 and a second anchor member 60 which can be inserted into the expansion catheter 11.

The expandable catheter 11 includes a long shaft portion 20, the balloon 30 provided at a distal portion of the shaft portion 20, and a hub 40 fixed to a base end of the shaft portion 20. The shaft portion 20 includes a first pipe body 21, a second pipe body 22 disposed inside the first pipe body 21, and a third pipe body 23 disposed outside the first pipe body 21. The second pipe body 22 is disposed coaxially with the first pipe body 21 inside the first pipe body 21. The second pipe body 22 extends distally relative to the first pipe body 21. The third pipe body 23 is fixed to an outer surface of the first pipe body 21 substantially parallel to the first tube body 21. The third pipe body 23 has a distal end located proximal of a distal end of the first pipe body 21.

The first pipe body 21 and the second pipe body 22 have an inflation lumen 24 formed therebetween. The inflation lumen 24 has an inflation fluid for inflating the balloon 30 flowing therethrough. The second pipe body 22 has a first wire lumen 25 formed inside. The first wire lumen 25 can be inserted with a guide wire and the first anchor member 50. The second pipe body 22 has a first distal opening portion 27 formed at a distal end. The first wire lumen 25 is opened in the first distal opening portion 27. The third pipe body 23 has a second wire lumen 26 formed inside. The second wire lumen 26 can be inserted with the second anchor member 60. The third pipe body 23 has a second distal opening portion 28 formed at the distal end. The second wire lumen 26 is opened in the second distal opening portion 28.

The hub 40 is fixed to proximal portions of the first pipe body 21, the second pipe body 22, and the third pipe body 23. The hub 40 has a first opening portion 41 that connects to (e.g., fluidly communicates with, etc.) the inflation lumen 24 between the first pipe body 21 and the second pipe body 22, a second opening portion 42 that connects to the first wire lumen 25 of the second pipe body 22, and a third opening portion 43 that connects to the second wire lumen 26 of the third pipe body 23. The first opening portion 41 functions as a port for flowing the inflation fluid into and out of the inflation lumen 24. By flowing the inflation fluid from the first opening portion 41, the inflation fluid flows into the balloon 30 via the inflation lumen 24. As a result, the balloon 30 can be inflated. The second opening portion 42 functions as a port for inserting or removing the guide wire and the first anchor member 50 into or from the first wire lumen 25. The third opening portion 43 functions as a port for inserting or removing the second anchor member 60 into or from the second wire lumen 26. In some embodiments, the proximal portion of the second pipe body 22 may be disposed distal of the hub 40, instead of inside and/or on the hub 40.

The first pipe body 21, the second pipe body 22, and the third pipe body 23 preferably have appropriate flexibility and appropriate rigidity. The first pipe body 21, the second pipe body 22, and the third pipe body 23 are each formed of a polymer material such as polyolefin (e.g., polyethylene, polypropylene, polybutene, ethylene-propylene copolymer, ethylene-vinyl acetate copolymer, ionomer, a mixture of two or more thereof, and the like), polyvinyl chloride, polyamide, polyamide elastomer, polyurethane, polyurethane elastomer, polyimide, fluororesin, a mixture thereof, a multilayer tube made of two or more of the above-described polymer materials, or the like.

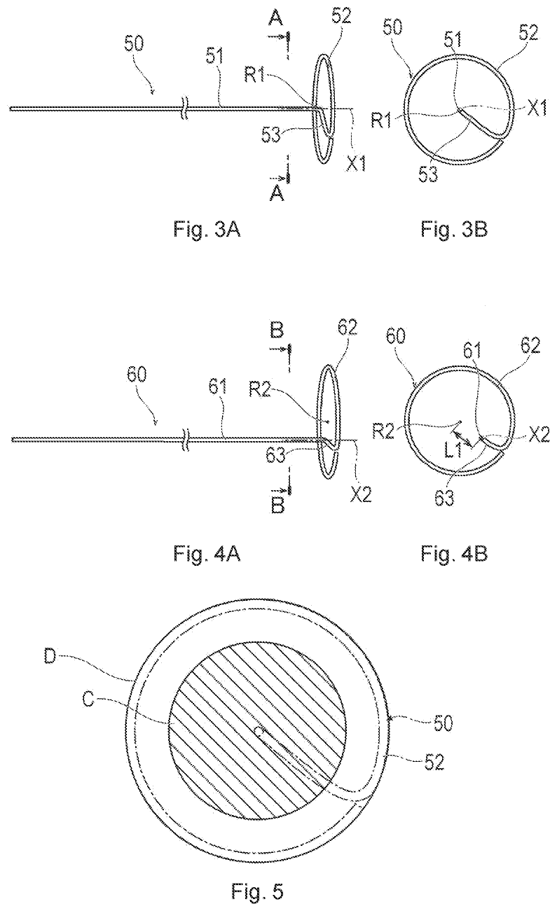

As illustrated in FIGS. 1, 3A, and 3B, the first anchor member 50 is a member disposed distally of the balloon 30 for holding the balloon 30 at a desired position. The first anchor member 50 can be inserted into the first wire lumen 25 from the second opening portion 42 of the hub 40 to protrude from the first distal opening portion 27 of the first wire lumen 25 disposed distally of the balloon 30. The first anchor member 50 is one elastically deformable wire. The first anchor member 50 includes a first proximal linear portion 51 having a substantially linear shape in a natural state without being applied with external force, a first ring portion 52 extending in a ring shape so as to draw a substantially perfect circle, and a first connection portion 53 disposed between the first proximal linear portion 51 and the first ring portion 52. The first proximal linear portion 51 may form a proximal end of or be disposed on a proximal side of the first anchor member 50, and the first ring portion 52 may form a distal end of or be disposed on a distal side of the first anchor member 50. In other words, the first proximal linear portion 51 may be arranged closer to a proximal end of the treatment device 10, while the first ring portion 52 may be arranged closer to the distal end of the treatment device 10. A virtual line X1 (which may be a centerline, etc.) passing through an axis of the first proximal linear portion 51 is substantially orthogonal to a plane in which the first ring portion 52 is located. The virtual line X1 passing through the axis of the first proximal linear portion 51 passes through a ring center R1 of the first ring portion 52. The first connection portion 53 has one end connected to an end portion of the first proximal linear portion 51 and the other end connected to an end portion of the first ring portion 52. The first connection portion 53 extends from the first proximal linear portion 51 to the first ring portion 52 along the plane in which the first ring portion 52 is located. However, it is to be understood that a shape of the first connection portion 53 is not particularly limited. The first anchor member 50 can be elastically deformed into a substantially linear shape as a whole, so as to be inserted into the first wire lumen 25 from the first opening portion 41. When the first anchor member 50 protrudes from the first distal opening portion 27 toward the distal side, the first ring portion 52 and the first connection portion 53 can be restored to original shapes by an elastic force thereof.

As illustrated in FIGS. 1, 4A, and 4B, the second anchor member 60 is a member proximal of the balloon 30 for holding the balloon 30 at a desired position. The second anchor member 60 can be inserted into the second wire lumen 26 from the third opening portion 43 of the hub 40 to protrude from the second distal opening portion 28 of the second wire lumen 26 disposed proximal of the balloon 30. The second anchor member 60 is one elastically deformable wire. The second anchor member 60 includes a second proximal linear portion 61 having a substantially linear shape in a natural state without being applied with external force, a second ring portion 62 extending so as to draw a substantially perfect circle, and a second connection portion 63 disposed between the second proximal linear portion 61 and the second ring portion 62. The second proximal linear portion 61 may form a proximal end of or be disposed on a proximal side of the second anchor member 60, and the second ring portion 62 may form a distal end of or be disposed on a distal side of the second anchor member 60. In other words, the second proximal linear portion may be arranged closer to a proximal end of the treatment device 10, while the second ring portion 62 may be arranged closer to the distal end of the treatment device 10. A virtual line X2 (which may be a centerline, etc.) passing through an axis of the second proximal linear portion 61 is substantially orthogonal to a plane in which the second ring portion 62 is located. The virtual line X2 passing through the axis of the second proximal linear portion 61 passes through a position deviated by a distance L1 from a ring center R2 of the second ring portion 62. The second connection portion 63 has one end connected to an end portion of the second proximal linear portion 61 and the other end connected to an end portion of the second ring portion 62. The second connection portion 63 extends from the second proximal linear portion 61 to the second ring portion 62 along the plane in which the second ring portion 62 is located. Note that a shape of the second connection portion 63 is not particularly limited. The second anchor member 60 can be elastically deformed into a substantially linear shape as a whole, so as to be inserted into the second wire lumen 26 from the second opening portion 42. When the second anchor member 60 protrudes from the second distal opening portion 28 toward the distal side, the second ring portion 62 and the second connection portion 63 can be restored to original shapes by an elastic force thereof. The ring center R2 of the second ring portion 62 is deviated by the distance L1 with respect to the virtual line X2 passing through the axis of the second proximal linear portion 61, while the distance L1 substantially coincides with a separated distance L2 (shown in FIG. 2) between an axis of the first wire lumen 25 and an axis of the second wire lumen 26 through which the second proximal linear portion 61 passes. Accordingly, the ring center R2 of the second ring portion 62 can substantially coincide with the axis of the first wire lumen 25. After the second anchor member 60 protrudes from the second wire lumen 26 to expand the second ring portion 62, it is desirable that rotation of the second anchor member 60 in the second wire lumen 26 is restricted so that the ring center R2 of the second ring portion 62 does not deviate from the axis of the first wire lumen 25. Accordingly, for example, the second distal opening portion 28 of the second wire lumen 26 may have a shape coinciding with a curved shape of the wire between the second proximal linear portion 61 and the second connection portion 63. As a result, the rotation of the second anchor member 60 with respect to the second wire lumen 26 can be restricted by disposing the wire between the second proximal linear portion 61 and the second connection portion 63 in an appropriate orientation in the second distal opening portion 28.

The first anchor member 50 and the second anchor member 60 are preferably each formed of, for example, a shape memory alloy imparted with a shape memory effect or superelasticity by heat treatment. A shape memory alloy, a Ni—Ti based alloy, a Cu—Al—Ni based alloy, a Cu—Zn—Al based alloy, or the like can be suitably used. In some examples, the constituent material of the first anchor member 50 and the second anchor member 60 is not particularly limited as long as the shapes thereof can be elastically restored, and may be, for example, other metals (including other alloys) such as stainless steel, resins, or the like. In addition, the first anchor member 50 and the second anchor member 60 may be formed of a plurality of materials. For example, the first anchor member 50 and the second anchor member 60 may have a resin material disposed around and/or outside of the above-described metal materials.

The balloon 30 is a member capable of inflating upon receiving an inflow of a fluid inside the balloon 30. The balloon 30 has a distal end fixed at a distal portion of the second pipe body 22 and a proximal end fixed at a distal portion of the first pipe body 21. Accordingly, the inside of the balloon 30 fluidically communicates with the inflation lumen 24.

The balloon 30 needs to have a certain degree of flexibility and to have a predetermined outer diameter when inflated. Accordingly, the balloon 30 is preferably made of a non-compliant material that does not excessively inflate even pressurized to a predetermined value or more, or a semi-compliant material that excessively inflates to some extent. Examples of the non-compliant material include polyethylene terephthalate and the like. Examples of the semi-compliant material include Nylon 6, Nylon 66, Nylon 12, and the like. The balloon 30 made of a material that does not excessively inflate or a material that excessively inflates to some extent may inflate to a desired outer diameter.

Note that the balloon 30 may have an outer diameter which can be adjusted by increasing the pressure. Such balloon 30 is made of, for example, a high-elasticity material. Examples of the high-elasticity material include various rubbers such as silicone rubber and latex rubber, polyurethane, polyamide, polyester, polystyrene-based thermoplastic elastomer, and the like.

The outer diameter of the balloon 30 when inflated is set to be less than an inner diameter of a blood vessel to be treated. For example, in the case where the blood vessel to be treated is a coronary artery, the coronary artery usually has an inner diameter of about 4 mm, and thus the outer diameter of the balloon 30 when inflated is preferably less than 4 mm.

The outer diameter of the balloon 30 when inflated is smaller than those of the radially expanded first anchor member 50 and second anchor member 60 so that the balloon 30 does not come into contact with an intravascular wall. Accordingly, as illustrated in FIG. 5, in a cross section orthogonal to an axis of the balloon 30, an area C occupied by the balloon 30 when inflated (area of a range surrounded by an outer surface of the balloon 30) is smaller than an area D of the first ring portion 52 of the first anchor member 50 when radially expanded and a range surrounded by the first ring portion 52.

Next, a method for treating acute myocardial infarction (AMI) using the above-described treatment device 10 will be described.

First, by percutaneous coronary intervention (PCI), a surgeon expands a lesion area in which stenosis or occlusion of the coronary artery has occurred with the balloon 30 to indwell a stent 100 in the lesion area. As a result, a state in which the lesion area is opened is maintained by the stent 100. By this indwelling operation of the stent 100, vascular endothelial cells of the lesion area are damaged and are almost peeled off, and substance permeability to the blood vessel wall of the lesion area is significantly improved. In addition, downstream of the site where stenosis or occlusion has occurred, due to exposure to ischemia, a gap junction between endothelial cells is broken, or the endothelial cells lose a barrier function or are peeled off, whereby the substance permeability is improved similarly.

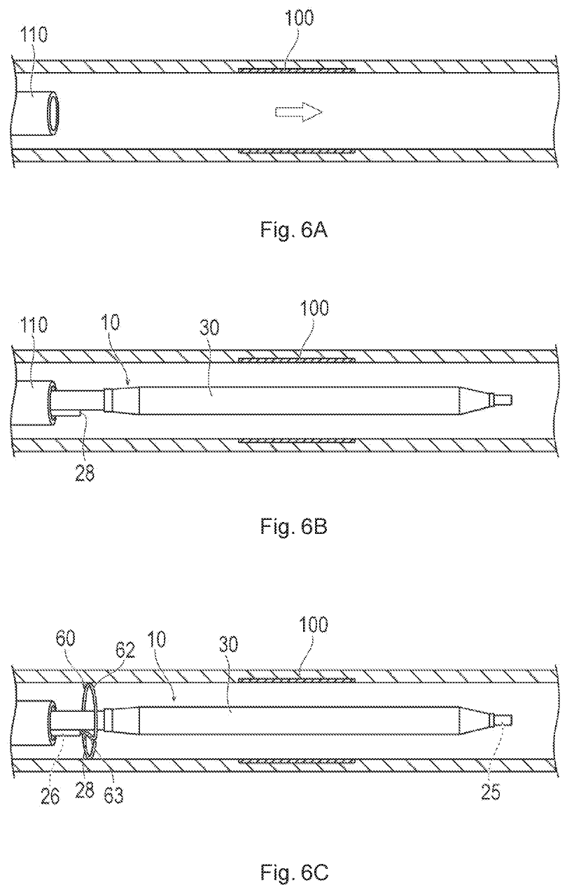

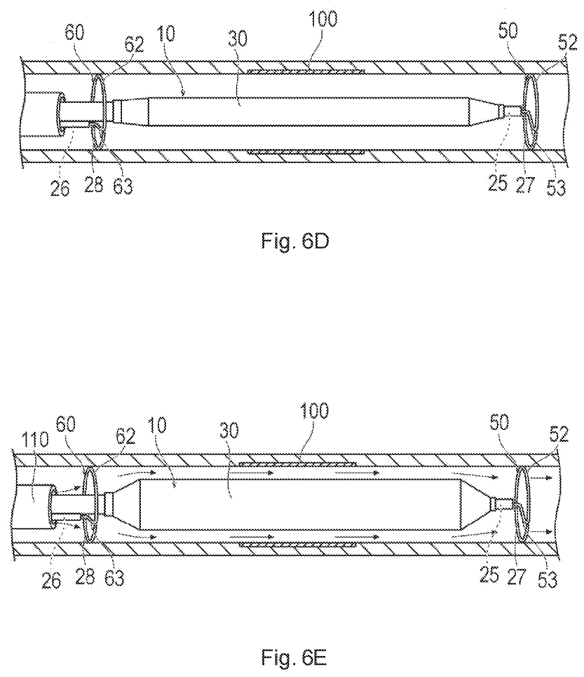

Next, as illustrated in FIG. 6A, the surgeon causes a catheter 110 to reach an upstream side of the lesion area, in which the stent 100 is indwelled, along a guide wire (not shown). Note that a white blank arrow indicates a direction of the blood flow. The catheter 110 may be the same one used in PCI. Next, the surgeon prepares the treatment device according to the present embodiment and inserts the same into the catheter 110 from a proximal side of the catheter 110. In the first wire lumen 25, the first anchor member 50 is disposed so as to not protrude from the first distal opening portion 27. In the second wire lumen 26, the second anchor member 60 is disposed so as to not protrude from the second distal opening portion 28. Next, as illustrated in FIG. 6B, the surgeon causes the treatment device 10 in which the balloon 30 has not been inflated to protrude from the catheter 110, and disposes the same inside the stent 100.

Next, as illustrated in FIG. 6C, the surgeon causes the second anchor member 60 to protrude from the second distal opening portion 28 of the second wire lumen 26. When the second anchor member 60 protrudes from the second wire lumen 26 toward the distal side, the second ring portion 62 and the second connection portion 63 return to the original shapes by a restoration force thereof. As a result, the second ring portion 62 is in contact with the intravascular wall over substantially 360 degrees. Accordingly, the second ring portion 62 is fixed to the blood vessel as an anchor member. The axis of the second wire lumen 26 through which the second anchor member 60 passes deviates from the axis of the first wire lumen 25 which substantially coincides with the axis of the balloon 30. However, as illustrated in FIG. 4B, the ring center R2 of the second ring portion 62 is deviated by the distance L1 with respect to the virtual line X2 passing through the axis of the second proximal linear portion 61, and the distance L1 substantially coincides with the separated distance L2 (shown in FIG. 2) between the axis of the first wire lumen 25 and the axis of the second wire lumen 26. Accordingly, the ring center R2 of the second ring portion 62 substantially coincides with the axis of the first wire lumen 25, that is, the axis of the balloon 30. After the second anchor member 60 protrudes from the second wire lumen 26 to expand the second ring portion 62, the rotation of the second anchor member 60 in the second wire lumen 26 is restricted so that the ring center R2 of the second ring portion 62 does not deviate from the axis of the first wire lumen 25.

Next, as illustrated in FIG. 6D, the surgeon causes the first anchor member 50 to protrude from the first distal opening portion 27 of the first wire lumen 25. When the first anchor member 50 protrudes from the first wire lumen 25 toward the distal side, the first ring portion 52 and the first connection portion 53 return to the original shapes by a restoration force thereof. As a result, the first ring portion 52 is in contact with the intravascular wall over substantially 360 degrees. Accordingly, the first ring portion 52 is fixed to the blood vessel as the anchor member. The axis of the first wire lumen 25 through which the first anchor member 50 passes substantially coincides with the axis of the balloon 30. Accordingly, the ring center of the first ring portion 52 substantially coincides with the axis of the first wire lumen 25 or, in other words, with the axis of the balloon 30. As a result, the surgeon can align the axis of the balloon 30 with a central axis of the blood vessel between the first anchor member 50 and the second anchor member 60, which are radially expanded and fixed to the blood vessel. At this time, the balloon 30 does not come into contact with the intravascular wall. Note that the surgeon may radially expand the first anchor member 50 before the second anchor member 60. Alternatively, the surgeon may radially expand one of the first anchor member 50 and the second anchor member 60 individually. In the long treatment device 10, a position of the distal end can become unstable due to the blood flow. Accordingly, it is preferable to at least use the first anchor member 50 to help maintain stability.

Next, the surgeon supplies the inflation fluid into the balloon 30 via the inflation lumen 24 from the third opening portion 43 of the hub 40. As a result, as illustrated in FIG. 6E, the balloon 30 is inflated. The outer diameter of the balloon 30 when inflated is smaller than the outer diameter of the first ring portion 52 of the first anchor member 50 and smaller than the outer diameter of the second ring portion 62 of the second anchor member 60. The outer diameter of the balloon 30 when inflated is smaller than an inner diameter of the blood vessel which is reopened by PCI and an inner diameter of the stent 100. Accordingly, the outer surface of the balloon 30 does not come into contact with the stent 100 and the blood vessel in which the stent 100 is indwelled. Therefore, the blood flow is not blocked by the balloon 30.

Next, the surgeon releases the physiologically active substance into the blood vessel via the catheter 110 inserted with the treatment device 10.

Any physiologically active substance to be injected into the blood vessel, sirolimus, everolimus, zotarolimus, biolimus, or the like, that has a cell proliferation inhibitory effect and is also an immune response inhibitor can be suitably used. In addition, as the physiologically active substance, anti-inflammatory agents such as dexamethasone can be suitably used. Furthermore, micro RNA (miRNA), nucleic acid, peptide, protein, or a mixture of two or more thereof can be suitably used as the physiologically active substance, from a viewpoint of regenerating the myocardium. For the purpose of slowly releasing the physiologically active substance for a long period of time, microparticles or nanoparticles that encapsulate such physiologically active substance may be used. When the physiologically active substance is cells, the physiologically active substance is taken in the blood vessel wall by flowing and rolling on the vascular endothelial cells while interacting with a specific adhesion factor. In addition, when the physiologically active substance is a substance having a medium molecular weight such as peptide, nucleic acid, and protein, the physiologically active substance is taken in the blood vessel wall while causing an electric or hydrophilic/hydrophobic interaction in the vicinity of surfaces of the vascular endothelial cells.

Note that the physiologically active substance is not limited to the above-described examples and may be a material having a bulge action such as collagen, hyaluronic acid, or alginate.

Incidentally, in the case where the balloon 30 is not disposed in the blood vessel, the blood flow is the fastest in a central portion of the blood vessel and the slowest in the vicinity of the blood vessel wall, as described in the Hagen-Poiseuille flow. Accordingly, when released to the blood vessel, the physiologically active substance is likely to be carried by the fast blood flow in the central portion of the blood vessel. Therefore, the physiologically active substance released to the blood vessel may hardly reach the vicinity of the blood vessel wall which has the highest shearing stress and is advantageous for taking in the substance, and an intake amount into a site to be treated (into the blood vessel wall) may be insufficient.

In some embodiments, the balloon 30 inflates at the central portion of the blood vessel without coming into contact with the blood vessel wall. Accordingly, the treatment device 10 can guide the physiologically active substance to the vicinity of the blood vessel wall by the balloon 30 while maintaining the blood flow reopened by the indwelling of the stent 100. As a result, the physiologically active substance released to the blood vessel can be carried by the blood flow to flow to the vicinity of the blood vessel wall which has a high shearing stress and is advantageous for taking in the substance. Accordingly, the physiologically active substance is effectively taken in a vascular tissue from the inner surface of the peripheral blood vessel including the lesion area, which is damaged and thus has an improved substance permeability, and gaps generated between the vascular endothelial cells. When the physiologically active substance is cells or genes that promote repair and/or regeneration of the myocardium, the physiologically active substance efficiently acts on the myocardium via the blood vessel having improved substance permeability (e.g., the ability of taking in substances). Therefore, the treatment device can effectively promote the repair and/or the regeneration of the myocardium damaged due to exposure to ischemia. Therefore, it is preferable that the outer diameter of the balloon 30, which is an inflatable structure, when inflated is smaller than the inner diameter of the blood vessel reopened by PCI, but is a diameter sufficient for reducing the blood flow in the central portion in the blood vessel and guiding the blood flow toward the blood vessel wall.

Even downstream of the site in which stenosis or occlusion has occurred, the physiologically active substance can be effectively taken in the vascular tissue to effectively act from the inner surface of the peripheral blood vessel including the lesion area, which is exposed to ischemia and thus has an improved substance permeability, and the gaps between the vascular endothelial cells.

As illustrated in FIG. 5, in the cross section orthogonal to the axis of the balloon 30, the area C occupied by the balloon 30 when inflated (area of the range surrounded by the outer surface of the balloon 30) is smaller than the area D of the first ring portion 52 of the first anchor member 50 when radially expanded and a range surrounded by the first ring portion 52. A ratio of the area C to the area D is not particularly limited, and is, for example, 10% to 80%, preferably 25% or more and less than 50%, and more preferably 30% to 45%. A smaller ratio of the area C to the area D can achieve a higher effect of maintaining the blood flow, but leads to a lower effect of guiding the blood flow to the vicinity of the blood vessel wall. A larger ratio of the area C to the area D can achieve a higher effect of guiding the blood flow to the vicinity of the blood vessel wall, but leads to a lower effect of maintaining the blood flow.

A length of the balloon 30 in an axial direction is not particularly limited, but is preferably equal to or greater than a length in the axial direction of the stent 100 when expanded and indwelled in the blood vessel, more preferably twice or more, and still more preferably three times or more in the case where the balloon 30 is used together with a treatment using the stent 100 as illustrated in FIG. 6E. When the length of the balloon 30 in the axial direction is equal to or greater than the length of the stent 100 in the axial direction, the balloon 30 can guide the blood flow to the vicinity of the blood vessel wall over the entire stent 100. When the length of the balloon 30 in the axial direction is twice or more the length of the stent 100 in the axial direction, the balloon 30 can pass through a range from upstream of the stent 100 to downstream of the stent 100 in addition to the stent 100. Accordingly, the balloon 30 can precisely guide the blood flow to the vicinity of the blood vessel wall over the entire stent 100. When the length of the balloon 30 in the axial direction is three times or more the length of the stent 100 in the axial direction, the balloon 30 can pass through a range of a length substantially the same as the stent 100 upstream of the stent 100 and a range of a length substantially the same as the stent 100 downstream of the stent 100 in addition to the stent 100. Accordingly, the balloon 30 can more precisely guide the blood flow to the vicinity of the blood vessel wall over the entire stent 100.

Note that the first anchor member 50 and/or the second anchor member 60 may be inserted in the expandable catheter 11 after the expandable catheter 11 has reached a target position of the coronary artery.

After release of a predetermined amount of the physiologically active substance is completed, the surgeon stops the release of the physiologically active substance. Next, as illustrated in FIG. 6D, the surgeon deflates the balloon 30. Next, as illustrated in FIG. 6C, the surgeon extracts the distal portion of the first anchor member 50, which is in a state of being radially expanded in the blood vessel, through the first wire lumen 25. Further, as illustrated in FIG. 6B, the surgeon extracts the distal portion of the second anchor member 60 radially expanded in the blood vessel through the second wire lumen 26. Note that the surgeon may extract the second anchor member 60 before the first anchor member 50. After the extraction, the surgeon extracts the treatment device 10, which is in a state of protruding into the blood vessel from the catheter 110, through the catheter 110. After this extraction, the surgeon removes the catheter 110 and the treatment device 10 from the blood vessel. As a result, the procedure is completed.

As described above, the treatment device 10 according to at least one embodiment of the present disclosure is the treatment device 10 for treating a lesion in a blood vessel, and includes: the shaft portion 20 including at least one lumen; the inflatable balloon 30 disposed at the distal side of the shaft portion 20; and at least one anchor member configured to be inserted into the lumen and to radially expand at a state in which the distal portion protrudes from the lumen. The balloon 30 has an outer diameter when inflated that is smaller than the outer diameter of the anchor member when radially expanded. When the anchor member radially expands in the blood vessel to come into contact with the blood vessel wall, the balloon 30 is held away from the blood vessel wall.

In the treatment device 10 configured as described above, when inflated, the balloon 30 can be maintained at a substantially central portion in the blood vessel without coming into contact with the blood vessel wall by causing the anchor member to come into contact with the blood vessel wall when radially expanded. As a result, the treatment device 10 can guide the physiologically active substance released from upstream of the balloon 30 to the vicinity of the blood vessel wall by the balloon 30 while maintaining the blood flow. Accordingly, the physiologically active substance released to the blood vessel can be carried by the blood flow to flow to the vicinity of the blood vessel wall which has a high shearing stress and is advantageous for taking in the substance. Therefore, the treatment device 10 can effectively guide the physiologically active substance to the vicinity of the blood vessel wall while maintaining the blood flow, thereby improving the permeability of the physiologically active substance to the blood vessel wall. For example, the treatment device 10 can inflate the balloon 30 in the blood vessel in which the site where stenosis or occlusion has occurred is opened and expanded. As a result, the treatment device 10 can effectively take the physiologically active substance in the vascular tissue from the vascular endothelial cells that have been damaged by being expanded and have improved substance permeability.

In addition, the anchor member may be formed of a shape memory alloy. As a result, the anchor member can be radially expanded by being restored to a memorized shape.

The first anchor member 50 is disposed at the distal side of the balloon 30 and the second anchor member 60 is disposed at the proximal side of the balloon 30. As a result, the first anchor member 50 and the second anchor member 60 can maintain the inflated balloon 30 at the substantially central portion in the blood vessel without causing the balloon 30 to come into contact with the blood vessel wall or inhibiting the inflation of the balloon 30. In the case where both the first anchor member 50 and the second anchor member 60 are provided, when inflated, the balloon 30 can be maintained at the substantially central portion in the blood vessel with high accuracy without coming into contact with the blood vessel wall.

The first wire lumen 25 passes through the center portion of the balloon 30 and opens in the distal side of the balloon 30. As a result, the first anchor member 50, which passes through the first wire lumen 25 opened in the distal side of the balloon 30, can radially expand at the distal side of the balloon 30.

The second wire lumen 26 extends along the axis of the balloon 30 and opens in the proximal side of the balloon 30. As a result, the second anchor member 60, which passes through the second wire lumen 26 opened in the proximal side of the balloon 30, can radially expand at the proximal side of the balloon 30.

The outer diameter of the balloon 30 when inflated may be less than 4 mm. As a result, even if the balloon 30 is inflated inside the coronary artery, which has an inner diameter of about 4 mm, the balloon may avoid contact with the blood vessel wall of the coronary artery, and thus is suitable for treatment of the coronary artery.

In addition, the invention also includes a treatment method for causing the physiologically active substance to act on the blood vessel wall in the blood vessel. The treatment method includes: an inflation step of inflating an inflatable inflation body in the blood vessel to dispose the inflation body at a position away from the blood vessel wall; and a guiding step of releasing the physiologically active substance from upstream of the inflation body in the blood vessel, thereby guiding the physiologically active substance toward the blood vessel wall by the inflation body.

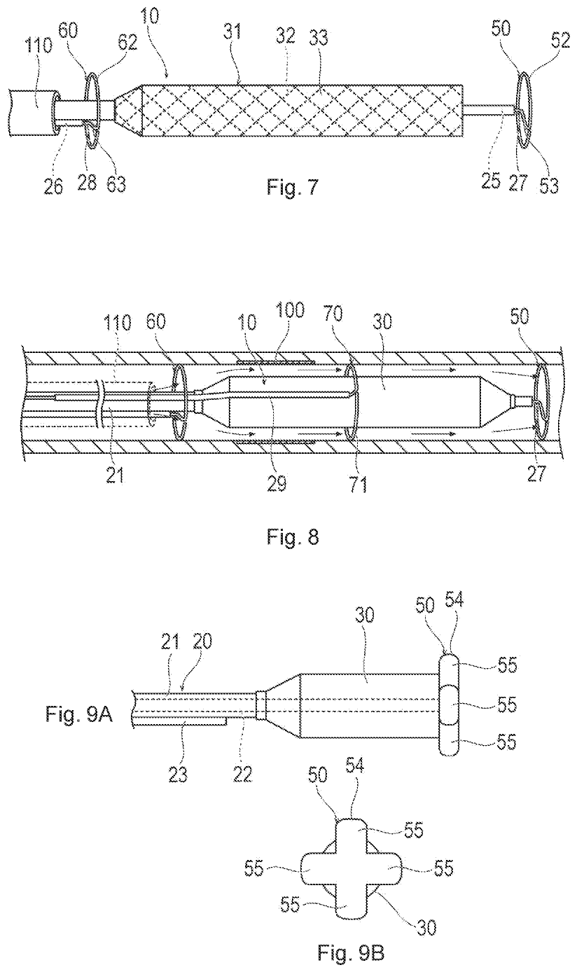

The treatment method configured as described above can maintain the blood flow because the inflation body does not block the blood vessel. The physiologically active substance released from upstream of the inflation body can be guided to the vicinity of the blood vessel wall by the inflation body, and thus the physiologically active substance can be carried by the blood flow to flow to the vicinity of the blood vessel wall which has a high shearing stress and is advantageous for taking in the substance. Therefore, the treatment method can effectively guide the physiologically active substance to the vicinity of the blood vessel wall while maintaining the blood flow, thereby improving the permeability of the physiologically active substance to the blood vessel wall. Note that the treatment method may not use the anchor member. In addition, the inflation body is not limited to the balloon 30. An inflation body 31 may, for example, have a covered stent-like shape in which gaps in a reticulate member 32 formed of a shape memory alloy or the like and capable of radially expanding in a cylindrical shape are covered with a film body 33, as in a first modification example illustrated in FIG. 7. The covered stent-like inflation body 31 can be inflated by a restoration force thereof by releasing the inflation body 31 from the catheter 110 or removing a sheath (not shown) stored in the inflation body 31 in advance. The physiologically active substance released from the catheter 110 is guided to the vicinity of the blood vessel by the film body 33 supported by the reticulate member 32. The film body 33 preferably does not have permeability, but may have permeability to some extent.

In addition, in the treatment method, the blood vessel is subjected to a treatment for expanding a lesion area in which stenosis or occlusion has occurred. The treatment method further includes a disposition step of radially expanding at least one anchor member capable of radially expanding to an outer diameter larger than that of the inflated inflation body, and disposing the at least one anchor member on a blood vessel wall upstream of and/or downstream of the lesion area in the blood vessel. As a result, in the treatment method, the anchor member can be disposed on the blood vessel wall at a position without inhibiting the inflation of the inflation body. Further, the anchor member used has an outer diameter larger than that of an outer diameter of the inflation body, and thus the inflated inflation body can be maintained at the substantially central portion of the blood vessel without coming into contact with the blood vessel wall. In the case where the anchor members are disposed at both the distal side and the proximal side, the anchor members can maintain the inflated inflation body at the substantially central portion in the blood vessel with high accuracy without causing the inflated inflation body to come into contact with the blood vessel wall.

In addition, the inflation step is performed after the disposition step. As a result, the inflation body can be inflated in a state in which the inflation body is held at an appropriate position by the disposition step. Accordingly, when the inflation body is inflated, the inflation body can be maintained at the substantially central portion of the blood vessel with high accuracy without coming into contact with the blood vessel wall.

In the disposition step of the treatment method, an axis of the inflation body is aligned with the central axis of the blood vessel by expanding the anchor member. As a result, when inflated, the balloon 30 can be maintained at the substantially central portion in the blood vessel with high accuracy without coming into contact with the blood vessel wall.

In the guiding step of the treatment method, the inflation body is held to not come into contact with the blood vessel wall. If the inflation body comes into contact with the blood vessel wall, the physiologically active substance cannot be guided to a part of the blood vessel wall, but by holding the inflation body to not come into contact with the blood vessel wall, the physiologically active substance can be effectively guided toward the blood vessel wall.

In the guiding step of the treatment method, the physiologically active substance is guided toward the blood vessel wall without blocking the blood flow by the inflation body. As a result, the blood vessel is not blocked by the inflation body, and thus the treatment method can prevent the downstream side from being in ischemic state, thereby improving safety.

Note that the invention is not limited to the embodiments described above, and various modifications can be made by those skilled in the art within a scope of the technical idea of the invention. For example, in the case where the lesion area of the blood vessel is long in the axial direction, a plurality of treatments may be continuously performed while deviating the position of the treatment device 10 in the axial direction.

In some examples, as illustrated in FIG. 8, the treatment device 10 may include a third anchor member 70 disposed circumferentially around the balloon 30 (or around an outer surface of the balloon 30). The third anchor member 70 disposed radially outward of the balloon 30 may be at least partially contained in a lumen of a fourth pipe body 29, and the fourth pipe body 29 may be partially disposed on the surface of the balloon 30 and partially disposed on a circumference surface of the first pipe body 21. The fourth pipe body 29 has an opening portion in the surface of the balloon 30. The fourth pipe body 29 has a proximal end located on the outer surface of the first pipe body 21, which may also be located on the hub 40. The third anchor member 70 can be expanded by protruding from the opening portion of the fourth pipe body 29 in the surface of the balloon 30. The expanded third anchor member 70 preferably has a third ring portion 71 whose ring center is located at the axis of the balloon 30. The third anchor member 70 is preferably in contact with the blood vessel wall at a position without coming into contact with the stent 100. That is, the third anchor member 70 is in contact with the blood vessel wall upstream of or downstream of the stent 100. The treatment device 10 provided with the third anchor member 70 disposed radially outward of the balloon 30 is effective in the case where the balloon 30 is long in the axial direction. The balloon 30 is likely to be bent and to come into contact with the blood vessel wall when being long in the axial direction. However, the treatment device 10 provided with the third anchor member 70 disposed radially outward of the balloon 30 can prevent the balloon 30 from coming into contact with the blood vessel wall by the third anchor member 70 even if the balloon 30 is long. For example, the treatment device 10 preferably includes the third anchor member 70 in the case where the length of the balloon 30 in the axial direction is four times or more the length in the axial direction of the stent 100 in a state of being expanded and indwelled in the blood vessel.

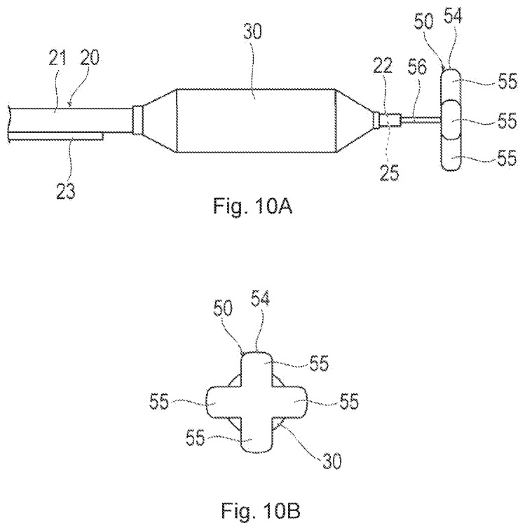

The form of the first anchor member 50 is not particularly limited as long as the first anchor member 50 can, in some examples, radially expand distally of the balloon 30. For example, the first anchor member 50 may include an anchor balloon 54 capable of inflating upon inflow of a fluid, as illustrated in FIGS. 9A and 9B. Note that the anchor balloon 54 is preferably fixed to the distal portion of the balloon 30 and inflated by being supplied with the fluid from a lumen different from the inflation lumen for inflating the balloon 30. For example, the anchor balloon 54 communicates with a lumen of the second pipe body 22, and can be inflated by being supplied with the fluid from the lumen of the second pipe body 22. In addition, the anchor balloon 54 has a non-circular circumference surface when viewed from the distal side, and has a plurality of protruding portions 55 protruding radially outward from the circumference surface of the balloon 30. The number of the protruding portions 55 is not particularly limited, and is preferably three or more, and four in the example shown in FIGS. 9A and 9B. The first anchor member 50 can come into contact with the blood vessel wall without blocking the blood flow by the anchor balloon 54 having the protruding portions 55. Alternatively, the first anchor member 50 includes the anchor balloon 54 and a long anchor shaft 56 provided with a lumen communicating with the inside of the anchor balloon 54, as illustrated in FIGS. 10A and 10B. The anchor balloon 54 can be inflated by being supplied with the fluid from the anchor shaft 56. The anchor balloon 54 can be deflated to be accommodated in the first wire lumen 25 of the second pipe body 22 together with the anchor shaft 56.

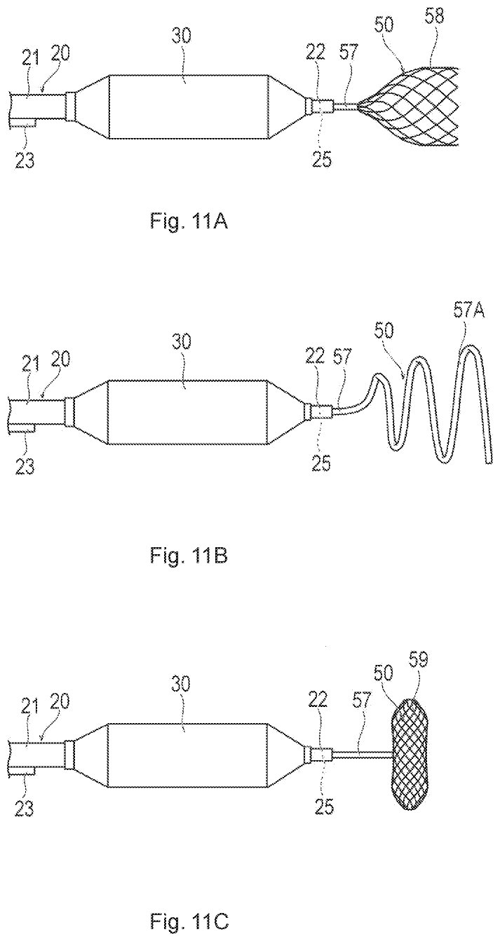

In some examples, the first anchor member 50 may include a reticulate member 58 formed of a shape memory alloy or the like and capable of radially expanding into a cylindrical shape and a long support shaft 57 supporting the member 58, as illustrated in FIG. 11A. The member 58 can be contracted to be accommodated in the first wire lumen 25 of the second pipe body 22 together with the support shaft 57.

In some examples, the first anchor member 50 may have a spiral body 57A formed of a shape memory alloy or the like and wound in a spiral shape and the long support shaft 57 supporting the spiral body 57A, as illustrated in FIG. 11B. The spiral body 57A can be linearly extended and contracted to be accommodated in the first wire lumen 25 of the second pipe body 22 together with the support shaft 57.

In some examples, the first anchor member 50 may include a mesh structure 59 knitted by a wire formed of a shape memory alloy or the like in a bag shape surrounding an internal space and the long support shaft 57 supporting the mesh structure 59, as illustrated in FIG. 11C. The mesh structure 59 can be linearly extended and contracted to be accommodated in the first wire lumen 25 of the second pipe body 22 together with the support shaft 57.

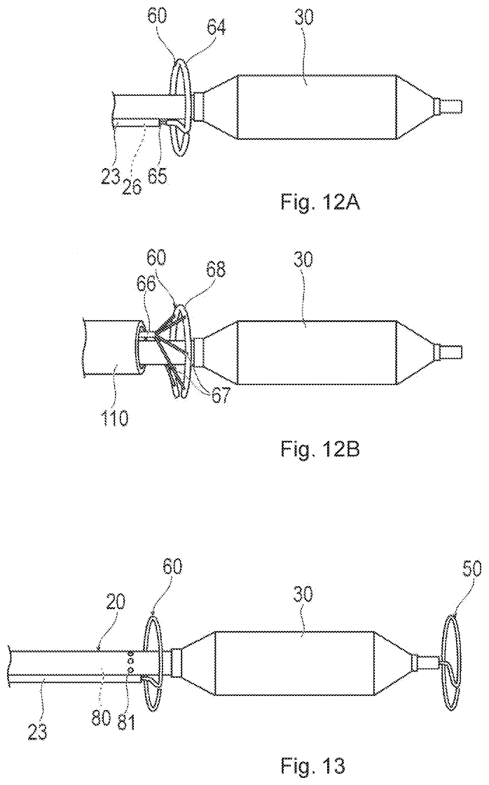

The form of the second anchor member 60 is not particularly limited as long as the second anchor member 60 can radially expand at the proximal side of the balloon 30. For example, the second anchor member 60 may include a ring-shaped anchor balloon 64 and an anchor shaft 65 provided with a lumen communicating with the inside of the anchor balloon 64, as illustrated in FIG. 12A. The anchor balloon 64 can be inflated by being supplied with the fluid from the anchor shaft 65. The anchor balloon 64 can be deflated to be accommodated in the second wire lumen 26 of the third pipe body 23 together with the anchor shaft 65.

In some examples, the second anchor member 60 may not be accommodated in the third pipe body 23 but inserted independently in the catheter 110 as illustrated in FIG. 12B. The second anchor member 60 may include a long support shaft 66, a plurality of branch shafts 67 branched from a distal portion of the support shaft 66, and an annular portion 68 supported by the branch shafts 67. The branch shafts 67 are formed of a shape memory alloy or the like. The annular portion 68 is a discontinuous ring body in which a part of 360 degrees is cut off. The annular portion 68 is formed of a material that is easily deformable. The second anchor member 60 can be accommodated in the catheter 110 together with the support shaft 66 by deforming the plurality of branch shafts 67 so as to approach each other while deforming the annular portion 68

In some examples, the treatment device 10 may be formed with a lumen 80 that releases the physiologically active substance into the blood vessel and at least one opening portion 81 as illustrated in FIG. 13. It is preferable to provide multiple opening portions 81 in order to allow the physiologically active substance to flow more uniformly over an entire circumference of the blood vessel wall. Further, it is generally considered that a larger diameter of the respective opening portions 81 or a larger number of the opening portions 81 achieve a lower injection resistance of a solution containing the physiologically active substance, which is advantageous for injection of a solution having a high viscosity.

The blood vessel treated by the treatment device 10 may be a blood vessel other than coronary artery. In a procedure performed before the treatment using the treatment device 10, the stent 100 may not be indwelled in the blood vessel. For example, in the procedure performed before the treatment using the treatment device 10, a procedure for expanding the lesion area may be performed by the balloon 30 without indwelling the stent 100. The treatment using the treatment device 10 is preferably performed continuously in the same surgery immediately after the procedure of expanding the lesion area, but may also be performed in another surgery after a certain period of time. In this case, it is desirable that damage to endothelial cells by the procedure for expanding the lesion area still remains, and the treatment using the treatment device 10 may be performed by another surgery within, for example, half a year. In the case where a silent rupture (asymptomatic collapse) of a vulnerable plaque (VP) (frangible atheroma) and a lesion area having a trace of self-healing are confirmed, such lesion area and a downstream area thereof are exposed to temporary ischemia, and thus lack of a barrier function of the endothelial cells and generate gaps between the endothelial cells, which increase the substance permeability into the vascular tissue. Therefore, the treatment using the treatment device 10 may be performed as a single surgery from a viewpoint of preventing sudden death due to a fatal VP rupture.

The treatment device 10 may be inserted from the downstream side of the blood vessel depending on the blood vessel to be treated. In this case, the physiologically active substance can be released from the first wire lumen 25.

What is claimed is:

1. A treatment device for treating a lesion in a blood vessel, the treatment device comprising:
a shaft portion including at least one lumen;
a balloon disposed at a distal side of the shaft portion and configured to inflate; and
a first anchor member configured to be inserted into the lumen and to radially expand when a distal portion of the first anchor member protrudes from the at least one lumen,
wherein the balloon has an outer diameter when inflated that is smaller than an outer diameter of the first anchor member when radially expanded, and
wherein, when the first anchor member radially expands in a blood vessel to come into contact with a blood vessel wall, the balloon is held away from the blood vessel wall.

2. The treatment device of claim 1, wherein the first anchor member comprises a shape memory alloy.

3. The treatment device of claim 1, wherein the treatment device further includes an anchor balloon configured to inflate upon inflow of a fluid.

4. The treatment device of claim 1, wherein the first anchor member is disposed on a distal side of the balloon.

5. The treatment device of claim 4, wherein the treatment device includes a second anchor member disposed circumferentially around the balloon.

6. The treatment device of claim 1, wherein the at least one lumen includes a first lumen that passes through a center portion of the balloon and includes an opening on a distal side of the balloon.

7. The treatment device of claim 6, wherein the at least one lumen includes a second lumen that extends along a first axis of the balloon and includes an opening on a proximal side of the balloon.

8. The treatment device according to claim 1, wherein the outer diameter of the balloon is less than 4 millimeters (mm) when inflated.

9. A treatment method for causing a physiologically active substance to act on a blood vessel wall in a blood vessel, the method comprising:
inflating an inflation body in the blood vessel to dispose the inflation body a first distance away from the blood vessel wall; and
guiding, using the inflation body, the physiological active substance released from an upstream side of the blood vessel relative to the inflation body toward the blood vessel wall.

10. The treatment method of claim 9, wherein the blood vessel is subjected to a treatment for expanding a lesion area in which at least one of stenosis and occlusion has occurred, and wherein the method further comprises:
radially expanding at least one anchor member capable of radially expanding to a first outer diameter larger than a second outer diameter of the inflation body when inflated, and
disposing the at least one anchor member on a blood vessel wall upstream of or downstream of the lesion area in the blood vessel.

11. The treatment method of claim 10, wherein the inflation body is inflated after the at least one anchor member is radially expanded.

12. The treatment method of claim 10, wherein an axis of the inflation body is aligned with a central axis of the blood vessel by expanding the at least one anchor member.

13. The treatment method of claim 12, wherein the inflation body is positioned so as to not come into contact with the blood vessel wall.

14. The treatment method of claim 13, wherein the inflation body does not block a blood flow when guiding the physiologically active substance toward the blood vessel wall.

15. A treatment device, comprising:
a first lumen;
an inflatable balloon disposed on a distal side of the first lumen, wherein the inflatable balloon has a first outer diameter when inflated; and
a first anchor member configured to be inserted into the first lumen and to radially expand to form a first ring with a second outer diameter when protruding from a distal portion of the first lumen,
wherein the second outer diameter is larger than the first outer diameter, and
wherein, when the first anchor member radially expands in a blood vessel to come into contact with a blood vessel wall, the inflatable balloon avoids contacting the blood vessel wall.

16. The treatment device of claim 15, wherein the first anchor member comprises a shape memory alloy.

17. The treatment device of claim 16, wherein the first anchor member includes at least one of a reticulate member and a mesh structure disposed on a distal end of the first anchor member.

18. The treatment device of claim 15, wherein the treatment device further comprises an anchor balloon configured to inflate upon receiving an inflow of a fluid.

19. The treatment device of claim 18, wherein the first anchor member is disposed on a distal side of the inflatable balloon.

20. The treatment device of claim 18, wherein the treatment device further comprises a second lumen that extends along a first axis of the inflatable balloon and includes an opening on a proximal side of the inflatable balloon.
